(19) United States
(12) Patent Application Publication
(10) Pub. No.: US 2024/0285916 A1
(43) Pub. Date: Aug. 29, 2024

(54) CONTROL DEVICE, INDEFLATION SYSTEM, AND INDEFLATION METHOD

(71) Applicant: Terumo Kabushiki Kaisha, Tokyo (JP)

(72) Inventors: Takumi FUKUDA, Hadano-shi (JP); Yoichiro KUWANO, Tokyo (JP); Kohei KANAYA, Fujisawa-shi (JP)

(73) Assignee: Terumo Kabushiki Kaisha, Tokyo (JP)

(21) Appl. No.: 18/435,239

(22) Filed: Feb. 7, 2024

(30) Foreign Application Priority Data
Feb. 28, 2023 (JP) 2023-030536

(51) Int. Cl. A61M 25/10

(52) U.S. Cl. CPC A61M 25/10187; A61M 2205/10; A61M 2205/3327; A61M 2205/3331; A61M 2205/50

(57) ABSTRACT

A control device configured to control a pressurization and decompression operation of a pump configured to inflate and deflate a balloon by pressurizing and decompressing the balloon attached to a distal portion of a catheter through the catheter inserted into a luminal organ of a patient includes a control unit configured to acquire image data obtained by imaging the luminal organ at least while the pump is caused to perform a pressurization operation, determine whether air bubbles are mixed in an expansion medium injected into the balloon in the pressurization operation with reference to the acquired image data, and when it is determined that the air bubbles are mixed, cause the pump to perform a decompression operation before a preset decompression timing.

### CROSS-REFERENCES TO RELATED APPLICATIONS

This application claims priority to Japanese Application No. 2023-030536 filed on Feb. 28, 2023, the entire content of which is incorporated herein by reference.

### TECHNOLOGICAL FIELD

The present disclosure generally relates to a control device, an indeflation system, and an indeflation method.

### BACKGROUND DISCUSSION

A balloon catheter inserted into a body has a balloon that inflates and deflates according to an injection amount of an expansion medium. Japanese Patent Application Publication No. 2002-159578 A, Japanese Patent Application Publication No. 2003-079738 A, and Japanese Patent Application Publication No. 2017-531460 A disclose an automatic indeflation device that automatically inflates and deflates a balloon.

The balloon catheter is internally primed with a fluid, such as a contrast agent or saline, prior to use. Air bubbles may remain inside the balloon catheter during priming. However, it is desirable to avoid the presence of air bubbles near a distal end of the balloon catheter, particularly inside the balloon.

### SUMMARY

The present disclosure makes it easier to immediately deal with a case where air bubbles are present inside the balloon.

Some aspects of the present disclosure are shown below.

[1] A control device configured to control a pressurization and decompression operation of a pump configured to inflate and deflate a balloon by pressurizing and decompressing the balloon attached to a distal portion of a catheter through the catheter inserted into a luminal organ of a patient, the control device including a control unit configured to acquire image data obtained by imaging the luminal organ at least while causing the pump to perform a pressurization operation, determine whether air bubbles are mixed in an expansion medium injected into the balloon in the pressurization operation with reference to the acquired image data, and when it is determined that the air bubbles are mixed, cause the pump to perform a decompression operation before a preset decompression timing.

[2] The control device according to claim [1], in which when it is determined that the air bubbles are mixed in the expansion medium, the control unit is configured to acquire a second image data obtained by imaging the luminal organ while causing the pump to decompress the balloon, compare the acquired second image data with first image data acquired while causing the pump to pressurize the balloon, confirm a change in size of the air bubbles mixed in the expansion medium, and cause the pump to perform the decompression operation before the preset decompression timing.

[3] The control device according to claim [2], in which when it is determined that the air bubbles are mixed in the expansion medium, after acquiring the second image data, the control unit is configured to acquire, as the first image data, image data obtained by imaging the luminal organ while causing the pump to pressurize the balloon.

[4] The control device according to claim [2], in which the first image data is image data referred to by the control unit when it is determined that the air bubbles are mixed in the expansion medium.

[5] The control device according to any one of [1] to [4], in which the control unit is configured to determine a possibility that air bubbles are mixed in the expansion medium by observing a change in pressure of the balloon while causing the pump to perform the pressurization operation, and decrease a speed of injecting the expansion medium into the balloon in the pressurization operation in a case where the determined possibility exceeds a reference.

[6] The control device according to claim [5], in which the control unit is configured to stop the pressurization operation when receiving a user operation requesting stop of the pressurization operation while causing the pump to perform the pressurization operation, and cause the pump to perform the decompression operation before the preset decompression timing even if it is not determined that the air bubbles are mixed in the expansion medium.

[7] The control device according to claim [5] or [6], in which the control unit is configured to refers to characteristic data that defines a characteristic of a change in intra-balloon pressure with respect to a change in intra-balloon medium amount for each balloon type, specify a characteristic according to a type of the balloon, and compare an observed change in pressure with respect to a change in an amount of the expansion medium in the pressurization operation with the specified characteristic to determine a possibility that the air bubbles are mixed in the expansion medium.

[8] The control device according to any one of [1] to [7], in which the image data includes data obtained by angiography.

[9] An indeflation system including: a control device according to any one of [1] to [8]; and the pump.

[10] A system including: a balloon attached to a distal end of a catheter, the catheter configured to be inserted into a luminal organ of a patient; a pump configured to inflate and deflate the balloon on the distal end of the catheter; a control device configured to cause the pump to perform a pressurization operation, acquire image data obtained by imaging the luminal organ during the pressurization operation, and determine whether air bubbles are mixed in an expansion medium injected into the balloon in the pressurization operation with reference to the acquired image data.

[11] An indeflation method comprising: inflating and deflating a balloon by pressurizing and decompressing, by means of a pump, the balloon attached to a distal portion of a catheter through the catheter inserted into a luminal organ of a patient; acquiring, by a control device, image data obtained by imaging the luminal organ at least while causing the pump to perform a pressurization operation, and determining whether air bubbles are mixed in an expansion medium injected into the balloon in the pressurization operation with reference to the acquired image data; and when the control device determines that the air bubbles are mixed in the expansion medium, causing the pump to perform a decompression operation before a preset decompression timing.

According to the present disclosure, it is easy to immediately deal with a case where air bubbles are present inside the balloon.

### BRIEF DESCRIPTION OF THE DRAWINGS

FIG. 1 is a diagram illustrating a configuration of an indeflation system according to an embodiment of the present disclosure.

FIG. 2 is a perspective view of a pump and an indeflator according to an embodiment of the present disclosure.

FIG. 3 is a schematic diagram of a balloon catheter according to an embodiment of the present disclosure.

FIG. 4 is a block diagram illustrating a configuration of a control device according to an embodiment of the present disclosure.

FIG. 5 is a flowchart illustrating an operation of the control device according to the embodiment of the present disclosure.

FIG. 6 is a diagram illustrating an example of an image captured by an imaging device according to an embodiment of the present disclosure.

FIG. 7 is a flowchart illustrating a modification of the operation of the control device according to the embodiment of the present disclosure.

FIG. 8 is a flowchart illustrating an additional operation of the control device according to the embodiment of the present disclosure.

FIG. 9 is a graph illustrating an example of characteristics of a balloon according to an embodiment of the present disclosure.

### DETAILED DESCRIPTION

Set forth below with reference to the accompanying drawings is a detailed description of embodiments of a control device, an indeflation system, and an indeflation method.

In the drawings, the same or corresponding portions are denoted by the same reference signs. In the description of the present embodiment, descriptions of the same or corresponding parts will be omitted or simplified as appropriate.

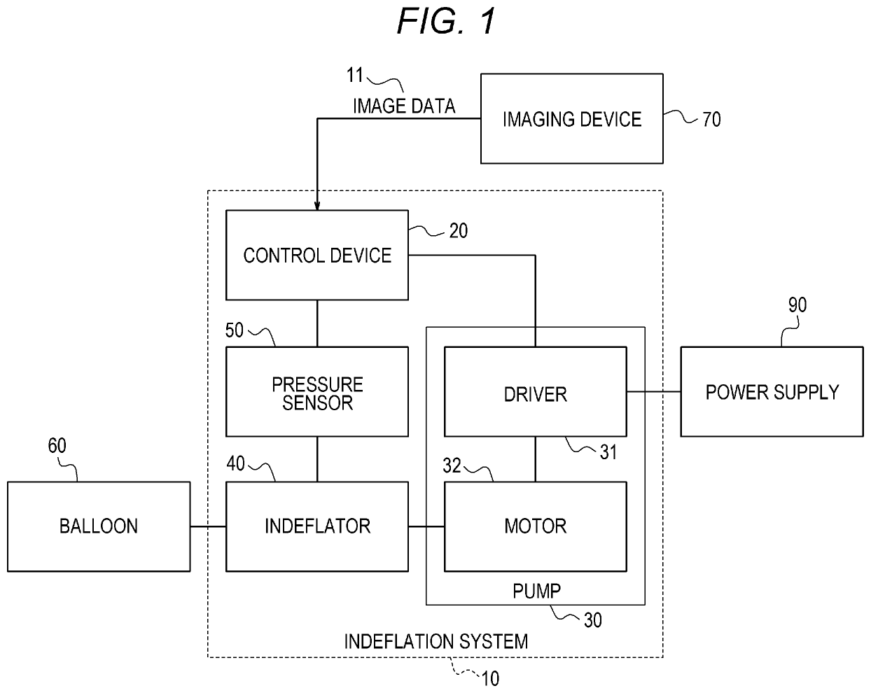

An outline of the present embodiment will be described with reference to FIG. 1.

A pump 30 inflates and deflates a balloon 60 by pressurizing and decompressing the balloon 60 attached to a distal portion such as a distal end of a catheter through the catheter inserted into a luminal organ of a patient. A control device 20 controls a pressurization and decompression operation of the pump 30. Specifically, the control device 20 acquires image data 11 obtained by imaging the luminal organ at least while causing the pump 30 to perform the pressurization operation, and determines whether air bubbles are mixed in an expansion medium injected into the balloon 60 by the pressurization operation with reference to the acquired image data 11. When determining that the air bubbles are mixed, the control device 20 causes the pump 30 to perform the decompression operation before the preset decompression timing. Therefore, according to the present embodiment, it is rather easy to immediately deal with a case where air bubbles are present inside the balloon 60. For example, an automatic indeflation operation can be immediately stopped, and an operator can resume the operation after removing the air bubbles from the balloon 60.

A configuration of an indeflation system 10 according to the present embodiment will be described with reference to FIG. 1.

The indeflation system 10 includes the control device 20, the pump 30, an indeflator (i.e., an inflation/deflation device) 40, and a pressure sensor 50.

The control device 20 is a computer that controls the pump 30. The pump 30 includes a driver 31 and a motor 32. The driver 31 is powered from a power supply 90 to drive the motor 32. The power supply 90 can be, for example, an external power supply such as a commercial power supply in the present embodiment, but may be a battery or may be provided in the indeflation system 10. The motor 32 is driven by the driver 31 to generate power. The indeflator 40 is an instrument for injecting an expansion medium such as a contrast agent or saline into the balloon 60. The expansion medium is automatically injected or discharged from the indeflator 40 to the balloon 60 by power generated from the motor 32. The control device 20 can adjust an injection amount of the expansion medium by controlling the driver 31 to adjust the power generated from the motor 32. The pressure sensor 50 is a sensor that measures a pressure inside the balloon 60. The control device 20 can adjust the pressure inside the balloon 60 to a desired pressure by adjusting the injection amount of the expansion medium with reference to a measurement value obtained from the pressure sensor 50.

The control device 20 can be connected to an imaging device 70 via, for example, a cable, wirelessly, or via a network such as a local area network (LAN). The imaging device 70 is an angiography device in the present embodiment, but may be a computed tomography (CT) device or a magnetic resonance imaging (MRI) device. In the present embodiment, the image data 11 can include data obtained by angiography.

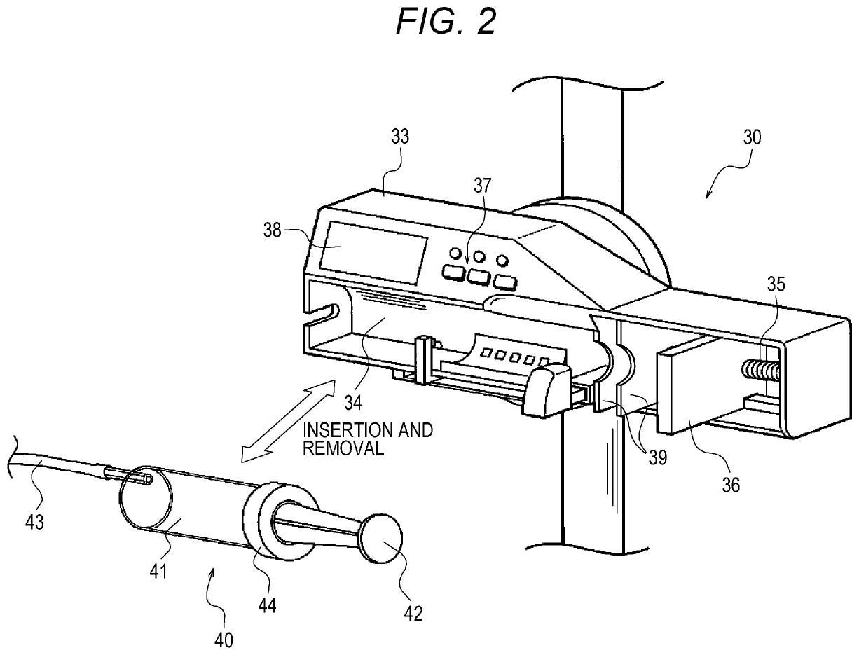

Specific configurations of the pump 30 and the indeflator 40 according to the present embodiment will be described with reference to FIG. 2.

The pump 30 includes a housing 33. The housing 33 is formed with a recess 34 for detachably accommodating the indeflator 40. A shaft 35 that converts rotational motion of the motor 32 into linear motion, a slider 36 supported by the shaft 35 so as to be capable of reciprocating, and a pair of plates 39 fixed to one end side of the shaft 35 are attached to the recess 34. The indeflator (inflation/deflation device) 40 can include a syringe 41 and a plunger 42. The syringe 41 includes a blade 44. When the slider 36 is moved by the driver 31 rotationally driving the motor 32 under the control of the control device 20 after the indeflator 40 is fitted in the recess 34 so as to fit the blade 44 of the syringe 41 between the pair of plates 39 and to close and fix the plunger 42 to the slider 36, the plunger 42 is pushed into the syringe 41, and the expansion medium in the syringe 41 is injected into the balloon 60 through a tube 43. As a result, the pressure of the balloon 60 increases and the balloon 60 inflates. On the other hand, when the driver 31 is controlled by the control device 20 to reversely rotate and drive the motor 32 to move the slider 36 in a reverse direction, the plunger 42 is pulled out to the outside of the syringe 41, and the expansion medium injected into the balloon 60 returns into the syringe 41 through the tube 43. As a result, the pressure of the balloon 60 decreases and the balloon 60 deflates.

The housing 33 of the pump 30 is further provided with a switch group 37 for performing an operation related to the operation of the pump 30 and a display 38 for displaying information related to the operation of the pump 30. The display 38 can be, for example, a liquid crystal display (LCD) or an organic electro luminescent (EL) display.

In the present embodiment, the control device 20 is accommodated in the housing 33 and integrated with the pump 30, but may be installed outside the housing 33 and connected to the pump 30, for example, via a cable, wirelessly, or via a network such as a LAN. In the present embodiment, the pressure sensor 50 is also integrated with the pump 30, but may be integrated with the indeflator 40, or may be installed separately from the pump 30 and the indeflator 40. The indeflator 40 may also be integrated into the pump 30, but as described above, is provided separately from the pump 30 in the present embodiment. Therefore, the indeflator 40 may be disposable.

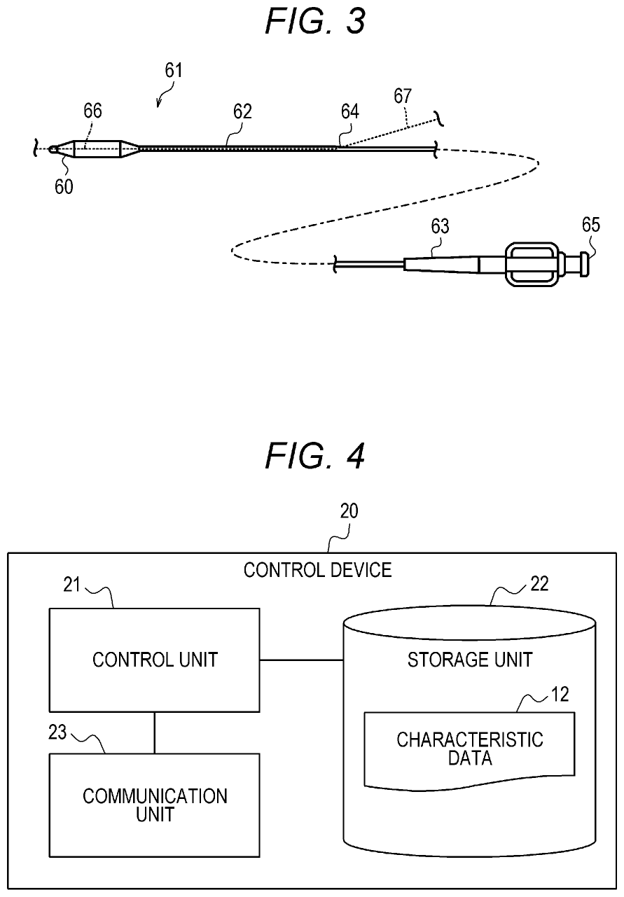

A specific configuration of a balloon catheter 61 which is a catheter according to the present embodiment will be described with reference to FIG. 3.

The balloon catheter 61 can include the balloon 60, a shaft 62, a hub 63, and a guide wire port 64.

The shaft 62 is connected to the hub 63 in a state where a lumen of the shaft 62 communicates with a flow path in the hub 63. A distal portion, such as a distal end of the shaft 62, is integrally joined with the balloon 60. The hub 63 includes a port 65 connectable to the indeflator 40. The balloon 60 inflates in a radial direction of the shaft 62 as an expansion medium is injected from the port 65 of the hub 63 through a lumen of the shaft 62. A guide wire lumen 66 through which a guide wire 67 can be inserted is formed in the lumen of the shaft 62. A proximal end of the guide wire lumen 66 communicates with the guide wire port 64.

A configuration of the control device 20 according to the present embodiment will be described with reference to FIG. 4.

The control device 20 includes a control unit 21, a storage unit 22, and a communication unit 23.

The control unit 21 includes at least one processor, at least one programmable circuit, at least one dedicated circuit, or any combination of the at least one processor, the at least one programmable circuit, and the at least one dedicated circuit. The processor can be a general-purpose processor such as a central processing unit (CPU) or a graphics processing unit (GPU), or a dedicated processor specialized for specific processing. The programmable circuit can be, for example, a field-programmable gate array (FPGA). The dedicated circuit can be, for example, an application specific integrated circuit (ASIC). The control unit 21 executes processing related to an operation of the control device 20 while controlling each unit of the control device 20.

The storage unit 22 can include at least one semiconductor memory, at least one magnetic memory, at least one optical memory, or any combination of the at least one semiconductor memory, the at least one magnetic memory, and the at least one optical memory. The semiconductor memory can be, for example, a random access memory (RAM), a read only memory (ROM), or a flash memory. The RAM can be, for example, a static random access memory (SRAM) or a dynamic random access memory (DRAM). The ROM can be, for example, an electrically erasable programmable read only memory (EEPROM). The flash memory can be, for example, a solid-state drive (SSD). The magnetic memory can be, for example, a hard disk drive (HDD). The storage unit 22 can function as, for example, a main storage, an auxiliary storage, or a cache memory. The storage unit 22 stores data used for the operation of the control device 20 and data obtained by the operation of the control device 20.

The communication unit 23 includes at least one communication module. The communication module is a module compatible with, for example, a wired LAN communication standard such as Ethernet®, a wireless LAN communication standard such as IEEE 802.11, a short-range wireless communication standard such as Bluetooth®, or a mobile communication standard such as long term evolution (LTE), a 4G standard, or a 5G standard. “IEEE” is an abbreviation for Institute of Electrical and Electronics Engineers. The “4G” is an abbreviation for 4th generation. The “5G” is an abbreviation for 5th generation. The communication unit 23 communicates with at least the pump 30, the pressure sensor 50, and the imaging device 70. The communication unit 23 receives data used for the operation of the control device 20 and transmits data obtained by the operation of the control device 20.

In the present embodiment, characteristic data 12 defining the characteristic of a change in the intra-balloon pressure with respect to a change in the intra-balloon medium amount for each balloon type is stored in the storage unit 22. The characteristic data 12 may be stored in the storage unit 22 in advance, or may be downloaded from an external server via the communication unit 23 and stored in the storage unit 22.

The function of the control device 20 is realized by executing a program according to the present embodiment by a processor as the control unit 21. That is, the function of the control device 20 can be realized by software. The program causes a computer to function as the control device 20 by causing the computer to execute the operation of the control device 20. That is, the computer functions as the control device 20 by executing the operation of the control device 20 according to the program.

The program can be stored in a non-transitory computer-readable medium. The non-transitory computer-readable medium can be, for example, a flash memory, a magnetic recording device, an optical disc, a magneto-optical recording medium, or a ROM. The program can be distributed, for example, by selling, transferring, or lending a portable medium, such as a secure digital card (SD card), a digital versatile disc (DVD), or a compact disc read only memory (CD-ROM), storing the program. The program may be distributed by storing the program in a storage of a server and transferring the program from the server to another computer. The program may be provided as a program product.

The computer temporarily stores, for example, a program stored in a portable medium or a program transferred from the server in the main storage. Then, the computer reads the program stored in the main storage by the processor and executes processing according to the read program by the processor. The computer may read the program directly from the portable medium and execute the processing according to the program. Each time the program is transferred from a server to a computer, the computer may sequentially execute processing according to the received program. The processing may be executed by what is called an application service provider (ASP) service in which the functions are implemented only by execution instructions and result acquisition instead of the program being transferred from the server to the computer. The programs include information that is used for processing by a computer and is equivalent to the programs. For example, data that is not a direct command to the computer but has a property that defines processing of the computer corresponds to the “information equivalent to the program”.

A part or all of the functions of the control device 20 may be realized by a programmable circuit or a dedicated circuit as the control unit 21. That is, some or all of the functions of the control device 20 may be realized by hardware.

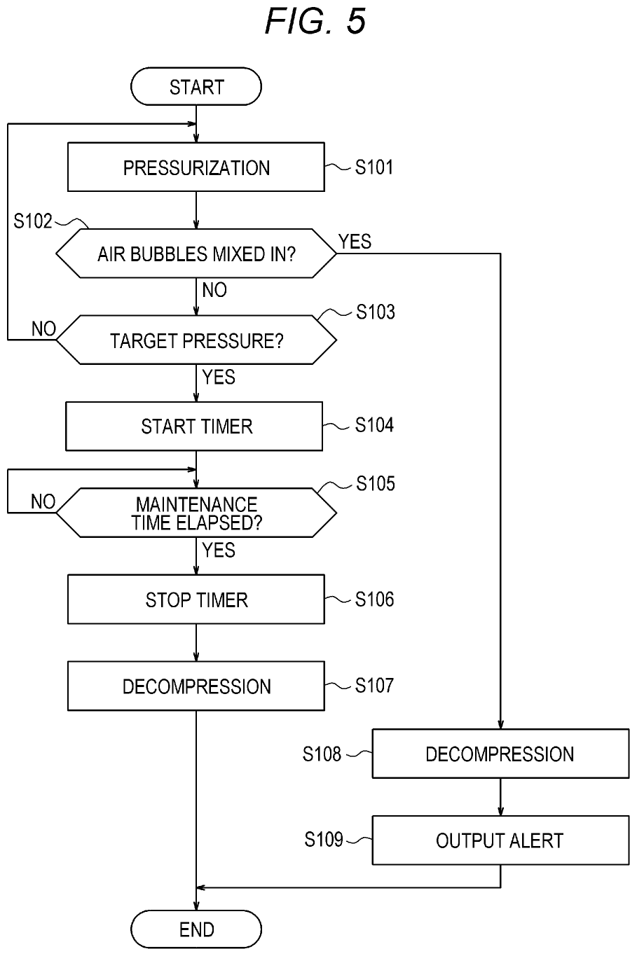

The operation of the control device 20 according to the present embodiment will be described with reference to FIG. 5. The operation illustrated in FIG. 5 corresponds to an automatic indeflation control method according to the present embodiment.

In S101, the control unit 21 of the control device 20 causes the pump 30 to perform a pressurization operation. Specifically, the control unit 21 of the control device 20 controls the pump 30 so that an expansion medium is injected from the indeflator 40 to the balloon 60 to pressurize the balloon 60.

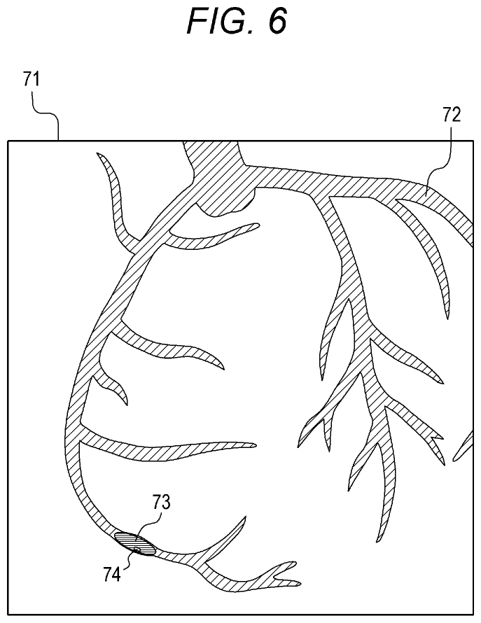

In S102, the control unit 21 of the control device 20 acquires the image data 11 from the imaging device 70 via the communication unit 23. The control unit 21 of the control device 20 refers to the acquired image data 11 to determine whether air bubbles are mixed in the expansion medium injected into the balloon 60 in the pressurization operation in S101. Specifically, the control unit 21 of the control device 20 acquires an angiographic image 71 as illustrated in FIG. 6 as the image data 11. For example, a blood vessel 72 of a patient becomes temporarily visible by X-rays when a contrast agent is provided in the blood vessel. In a case where the expansion medium has a contrast property (i.e., a radiopaque property), when the balloon 60 is filled with the expansion medium by the pressurization operation in S101, the shape of the balloon 60 becomes visible by X-rays. Since the air bubbles are not imaged, when there is a portion that is not imaged in the visible balloon 60, the portion can be estimated to be the air bubble. The control unit 21 of the control device 20 extracts an image region 73 corresponding to a position where the balloon 60 is disposed in the blood vessel 72 from the angiographic image 71. The control unit 21 of the control device 20 analyzes the extracted image region 73 by image processing, and determines whether a pixel group 74 corresponding to the air bubbles are included in the image region 73, thereby determining whether the air bubbles are mixed in the expansion medium. An arbitrary method may be used for the image processing, but for example, a method of detecting an edge of an air bubble in the image region 73 can be used. Machine learning such as deep learning may be used.

In a case where it is determined in S102 that air bubbles are mixed in the expansion medium, processing of S108 is executed. In a case where it is determined in S102 that air bubbles are not mixed in the expansion medium, processing of S103 is executed.

In S103, the control unit 21 of the control device 20 determines whether the pressure of the balloon 60 has reached a preset target pressure with reference to the measurement value obtained from the pressure sensor 50. The target pressure may be set for each balloon type or may be arbitrarily set by an operator. The control unit 21 of the control device 20 continues the pressurization operation in S101 until it is determined that the pressure of the balloon 60 has reached the target pressure. In a case where the pressure of the balloon 60 exceeds the target pressure, a decompression operation may be performed. While the pressurization or decompression operation is being continued, the processing of S102 is also repeatedly executed. When determining that the pressure of the balloon 60 has reached the target pressure, the control unit 21 of the control device 20 starts a timer in S104.

In S105, the control unit 21 of the control device 20 determines whether a maintenance time set in advance as a desired time from when the pressure of the balloon 60 reaches the target pressure to when the decompression operation of the pump 30 is started has elapsed. The maintenance time is desirably within a range of 1 second or more and 30 seconds or less, and can be, for example, 10 seconds. The maintenance time may be set for each balloon type or may be arbitrarily set by the operator. The control unit 21 of the control device 20 does not start the decompression operation in described later and maintains the pressure of the balloon 60 at the target pressure until it is determined that the maintenance time has elapsed. At this time, when there is a change in the pressure of the balloon 60, a pressurization or decompression operation may be performed so that the target pressure can be maintained. When determining that the maintenance time has elapsed, the control unit 21 of the control device 20 stops the timer in S106.

In both S107 and S108, the control unit 21 of the control device 20 causes the pump 30 to perform the decompression operation. Specifically, the control unit 21 of the control device 20 controls the pump 30 so that the expansion medium is sucked (i.e., suctioned) from the balloon 60 to the indeflator and the balloon 60 is decompressed. In S107, the decompression operation is performed when the maintenance time elapses, that is, at a decompression timing set in advance. In S108, the decompression operation is performed before the maintenance time elapses and at the time of air bubble detection, that is, before the preset decompression timing.

After S108, in S109, the control unit 21 of the control device 20 outputs an alert. Specifically, the control unit 21 of the control device 20 causes the display 38 of the pump 30 to display an alert message indicating that air bubbles are mixed in the expansion medium inside the balloon 60. Instead of or in addition to the display of the alert message, output of an alert sound, lighting of an alert lamp, or both of the output of the alert sound and the lighting of the alert lamp may be performed.

The operation illustrated in FIG. 5 ends after any one of the processing in S107 and the processing in S109 is executed.

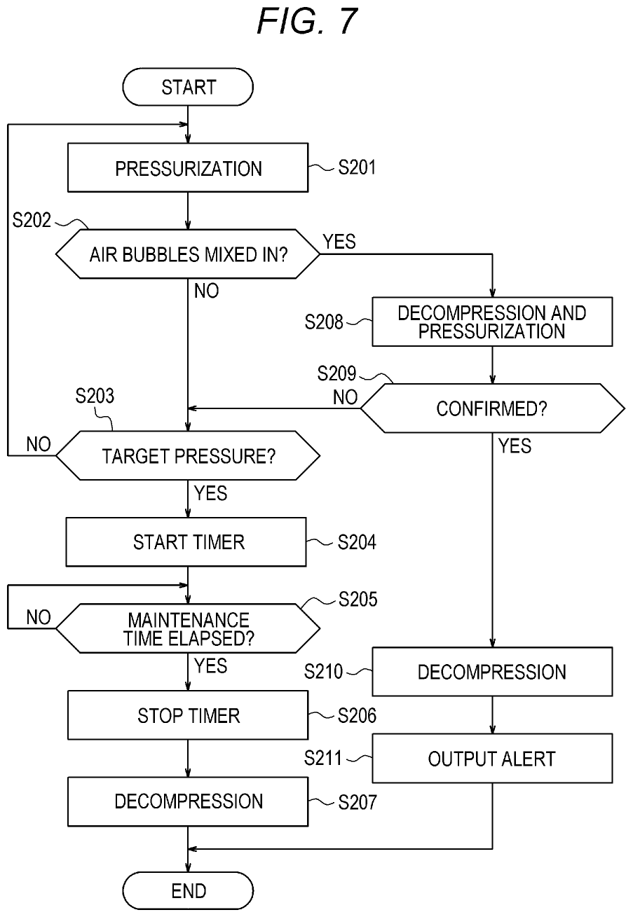

A modified example of the operation of the control device 20 according to the present embodiment will be described with reference to FIG. 7.

Since the processing from S201 to S207 is similar to the processing from S101 to S107 in FIG. 5, the description of S201 to S207 is omitted.

In a case where it is determined in S202 that air bubbles are mixed in the expansion medium, in S208, the control unit 21 of the control device 20 acquires second image data obtained by imaging the luminal organ of the patient from the imaging device 70 via the communication unit 23 while causing the pump 30 to decompress the balloon 60. In S209, the control unit 21 of the control device 20 compares the second image data acquired in S208 with the first image data acquired while the balloon 60 is pressurized by the pump 30 to confirm a change in size of the air bubble mixed in the expansion medium. In a case where the air bubbles become larger at the time of pressure reduction and smaller at the time of pressure increase, the determination result in S202 that the air bubbles are mixed in the expansion medium is correct, but otherwise, the determination result in S202 may be incorrect.

The first image data may be the image data 11 referred to by the control unit 21 of the control device 20 when it is determined that air bubbles are mixed in the expansion medium, that is, the image data 11 acquired in S202. However, in the present embodiment, the first image data is newly acquired from the imaging device 70. That is, in S208, after acquiring the second image data, the control unit 21 of the control device 20 acquires new image data obtained by imaging the luminal organ of the patient as the first image data from the imaging device 70 via the communication unit 23 while causing the pump 30 to pressurize the balloon 60. The decompression and the pressurization may be alternately repeated twice or more. That is, the second image data and the first image data may be alternately acquired twice or more. In a case where the air bubbles increase every time the pressure is reduced and decrease every time the pressure is increased, the determination result of S202 is correct, but otherwise, the determination result of S202 may be incorrect.

In a case where it has been confirmed in S209 that the determination result of S202 is correct, processing of S210 is executed. In a case where it has not been confirmed in S209 that the determination result of S202 is correct, the processing of S203 is executed.

Since the processing in S210 and S211 is similar to the processing in S108 and S109 in FIG. 5, the description of S210 and S211 is omitted.

The operation illustrated in FIG. 7 ends after any one of the processing in S207 and the processing in S211 is executed. According to the operation illustrated in FIG. 7, it can be relatively easy to avoid cancellation or delay of the surgery due to erroneous determination that air bubbles are mixed in the expansion medium.

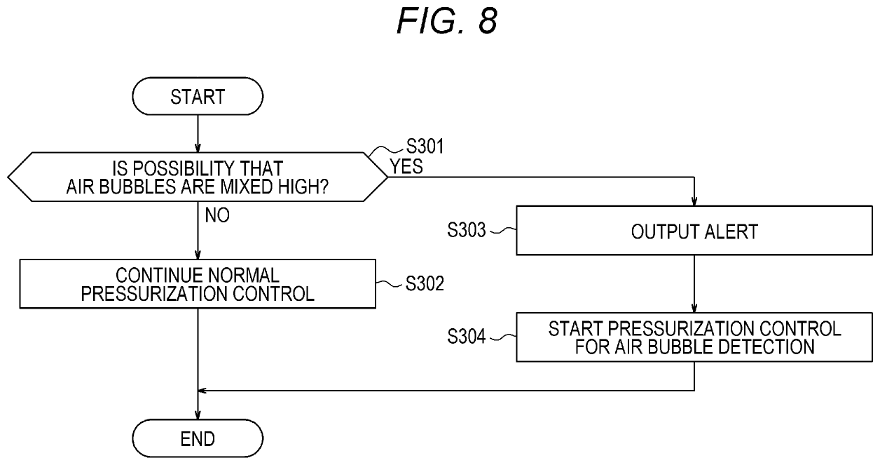

An additional operation of the control device 20 according to the present embodiment will be described with reference to FIG. 8. In a case where it is determined in S102 of FIG. 5 or S202 of FIG. 7 that no air bubbles are mixed in the expansion medium, the operation illustrated in FIG. 8 may be additionally performed.

In S301, the control unit 21 of the control device 20 monitors the measurement values obtained from the pressure sensor 50 in time series to observe a change in the pressure of the balloon 60 and determine the possibility that air bubbles are mixed in the expansion medium. Specifically, the control unit 21 of the control device 20 acquires the characteristic data 12 from the storage unit 22. The control unit 21 of the control device 20 refers to the acquired characteristic data 12 to specify the characteristic of a change in the intra-balloon pressure with respect to a change in the intra-balloon medium amount according to the type of the balloon 60. The control unit 21 of the control device 20 compares the observed change in pressure with respect to the change in the amount of the expansion medium in the pressurization operation in S101 of FIG. 5 or S201 of FIG. 7 with the specified characteristic to determine the possibility that the air bubbles are mixed in the expansion medium. Here, the fact that “the possibility that air bubbles are mixed in the expansion medium” is relatively high may mean that the probability that air bubbles are mixed in the expansion medium is relatively high, or may mean that the amount of air bubbles estimated to be mixed in the expansion medium is relatively large.

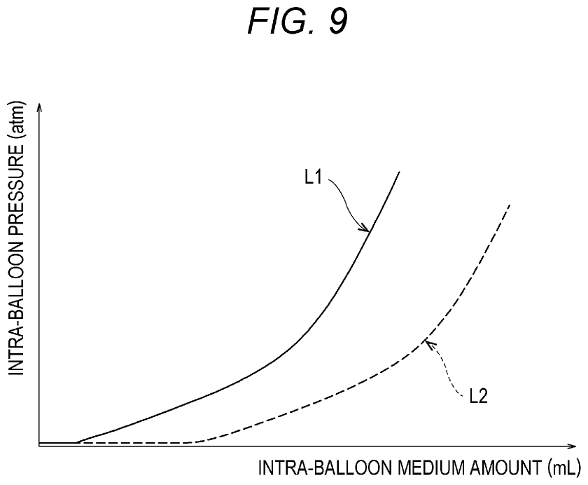

As illustrated in FIG. 9, the characteristic of the change in the intra-balloon pressure with respect to the change in the intra-balloon medium amount is determined by a reference line L1 for each balloon type, but when air bubbles are mixed in the expansion medium, deviation from the reference line L1 occurs as indicated by a curve L2. In the operation illustrated in FIG. 8, it is possible to determine the possibility that air bubbles are mixed in the expansion medium by detecting that the deviation is larger than a predetermined reference.

In a case where the possibility determined in S301 exceeds the standard, that is, in a case where it is determined that the possibility that air bubbles are mixed in the expansion medium is high, processing of S303 is executed. In a case where the possibility determined in S301 does not exceed the standard, that is, in a case where it is determined that the possibility that air bubbles are mixed in the expansion medium is relatively low, processing of S302 is executed.

In S302, the control unit 21 of the control device 20 continues the normal pressurization control. That is, the control unit 21 of the control device 20 maintains a speed at which the expansion medium is injected into the balloon 60 at the current speed in the pressurization operation in S101 in FIG. 5 or S201 in FIG. 7.

In S303, the control unit 21 of the control device 20 outputs an alert. Specifically, the control unit 21 of the control device 20 causes the display 38 of the pump 30 to display an alert message indicating that there is a relatively high possibility that air bubbles are mixed in the expansion medium inside the balloon 60. Instead of or in addition to the display of the alert message, output of an alert sound, lighting of an alert lamp, or both of the output of the alert sound and the lighting of the alert lamp may be performed.

After S303, in S304, the control unit 21 of the control device 20 starts pressurization control for air bubble detection. That is, the control unit 21 of the control device 20 decreases a speed of injecting the expansion medium into the balloon 60 by the pressurization operation in S101 in FIG. 5 or 201 in FIG. 7. As a result, it is possible to secure a relatively long time for the operator to determine whether to stop the pressurization operation.

The control unit 21 of the control device 20 may receive a user operation requesting stop of the pressurization operation while causing the pump 30 to perform the pressurization operation. As a switch for performing such a user operation, an emergency stop switch may be included in the switch group 37 of the pump 30. In response to a user operation, the control unit 21 of the control device 20 may stop the pressurization operation and cause the pump 30 to perform the decompression operation before the preset decompression timing even if it is not determined that the air bubbles are mixed in the expansion medium.

As described above, in the present embodiment, there is used the indeflation method including: inflating and deflating the balloon 60 by pressurizing and decompressing the balloon 60 by the pump 30; acquiring the image data 11 at least while causing the pump 30 to perform the pressurization operation by the control device 20, and determining whether air bubbles are mixed in the expansion medium injected into the balloon 60 by the pressurization operation with reference to the acquired image data 11; and when determining that air bubbles are mixed in the expansion medium by the control device 20, causing the pump 30 to perform the decompression operation before the preset decompression timing. Therefore, according to the present embodiment, it is relatively easy to immediately deal with a case where air bubbles are present inside the balloon 60.

The present disclosure is not limited to the above-described embodiment. For example, two or more blocks described in the block diagrams may be integrated, or one block may be divided. Instead of executing a plurality of steps described in the flowchart in time series according to the description, the steps may be executed in parallel or in a different order according to the processing capability of the device that executes each step or as necessary. In addition, modifications can be made within a scope not departing from the gist of the present disclosure.

The detailed description above describes embodiments of a control device, an indeflation system, and an indeflation method. The invention is not limited, however, to the precise embodiments and variations described. Various changes, modifications and equivalents may occur to one skilled in the art without departing from the spirit and scope of the invention as defined in the accompanying claims. It is expressly intended that all such changes, modifications and equivalents which fall within the scope of the claims are embraced by the claims.

What is claimed is:

1. A control device configured to control a pressurization and decompression operation of a pump configured to inflate and deflate a balloon by pressurizing and decompressing the balloon attached to a distal portion of a catheter through the catheter inserted into a luminal organ of a patient, the control device comprising:
a control unit configured to acquire image data obtained by imaging the luminal organ at least while causing the pump to perform a pressurization operation, determine whether air bubbles are mixed in an expansion medium injected into the balloon in the pressurization operation with reference to the acquired image data, and when it is determined that the air bubbles are mixed, cause the pump to perform a decompression operation before a preset decompression timing.

2. The control device according to claim 1, wherein when it is determined that the air bubbles are mixed in the expansion medium, the control unit is configured to acquire a second image data obtained by imaging the luminal organ while causing the pump to decompress the balloon, compare the acquired second image data with first image data acquired while causing the pump to pressurize the balloon, confirm a change in size of the air bubbles mixed in the expansion medium, and cause the pump to perform the decompression operation before the preset decompression timing.

3. The control device according to claim 2, wherein when it is determined that the air bubbles are mixed in the expansion medium, after acquiring the second image data, the control unit is configured to acquire, as the first image data, image data obtained by imaging the luminal organ while causing the pump to pressurize the balloon.

4. The control device according to claim 2, wherein the first image data is image data referred to by the control unit when it is determined that the air bubbles are mixed in the expansion medium.

5. The control device according to claim 1, wherein the control unit is configured to determine a possibility that air bubbles are mixed in the expansion medium by observing a change in pressure of the balloon while causing the pump to perform the pressurization operation, and decrease a speed of injecting the expansion medium into the balloon in the pressurization operation in a case where the determined possibility exceeds a reference.

6. The control device according to claim 5, wherein the control unit is configured to stop the pressurization operation when receiving a user operation requesting stop of the pressurization operation while causing the pump to perform the pressurization operation, and cause the pump to perform the decompression operation before the preset decompression timing even if it is not determined that the air bubbles are mixed in the expansion medium.

7. The control device according to claim 5, wherein the control unit is configured to refer to characteristic data that defines a characteristic of a change in intra-balloon pressure with respect to a change in intra-balloon medium amount for each balloon type, specify a characteristic according to a type of the balloon, and compare an observed change in pressure with respect to a change in an amount of the expansion medium in the pressurization operation with the specified characteristic to determine a possibility that the air bubbles are mixed in the expansion medium.

8. The control device according to claim 1, wherein the image data includes data obtained by angiography.

9. An indeflation system comprising:
a control device according to claim 1; and
the pump.

10. A system comprising:
a balloon attached to a distal end of a catheter, the catheter configured to be inserted into a luminal organ of a patient;
a pump configured to inflate and deflate the balloon on the distal end of the catheter;
a control device configured to cause the pump to perform a pressurization operation, acquire image data obtained by imaging the luminal organ during the pressurization operation, and determine whether air bubbles are mixed in an expansion medium injected into the balloon in the pressurization operation with reference to the acquired image data.

11. The system according to claim 10, wherein the control device is configured to cause the pump to perform a decompression operation before a preset decompression timing when the control device determines that the air bubble are mixed in the expansion medium being injected into the balloon during the pressurization operation.

12. The system according to claim 11, wherein when it is determined that the air bubbles are mixed in the expansion medium, the control device is configured to acquire a second image data obtained by imaging the luminal organ while causing the pump to decompress the balloon, compare the acquired second image data with first image data acquired while causing the pump to pressurize the balloon, confirm a change in size of the air bubbles mixed in the expansion medium, and cause the pump to perform the decompression operation before the preset decompression timing; and
wherein when it is determined that the air bubbles are mixed in the expansion medium, after acquiring the second image data, the control unit is configured to acquire, as the first image data, image data obtained by imaging the luminal organ while causing the pump to pressurize the balloon.

13. The system according to claim 11, wherein the first image data is image data referred to by the control device when it is determined that the air bubbles are mixed in the expansion medium.

14. The system according to claim 11, wherein the control device is configured to determine a possibility that air bubbles are mixed in the expansion medium by observing a change in pressure of the balloon while causing the pump to perform the pressurization operation, and decrease a speed of injecting the expansion medium into the balloon in the pressurization operation in a case where the determined possibility exceeds a reference; and
stop the pressurization operation when receiving a user operation requesting stop of the pressurization operation while causing the pump to perform the pressurization operation, and cause the pump to perform the decompression operation before the preset decompression timing even if it is not determined that the air bubbles are mixed in the expansion medium.

15. The system according to claim 14, wherein the control device is configured to refer to characteristic data that defines a characteristic of a change in intra-balloon pressure with respect to a change in intra-balloon medium amount for each balloon type, specify a characteristic according to a type of the balloon, and compare an observed change in pressure with respect to a change in an amount of the expansion medium in the pressurization operation with the specified characteristic to determine a possibility that the air bubbles are mixed in the expansion medium.

16. An indeflation method comprising:
inflating and deflating a balloon by pressurizing and decompressing, by means of a pump, the balloon attached to a distal portion of a catheter through the catheter inserted into a luminal organ of a patient;
acquiring, by a control device, image data obtained by imaging the luminal organ at least while causing the pump to perform a pressurization operation, and determining whether air bubbles are mixed in an expansion medium injected into the balloon in the pressurization operation with reference to the acquired image data; and
when the control device determines that the air bubbles are mixed in the expansion medium, causing the pump to perform a decompression operation before a preset decompression timing.

17. The indeflation method according to claim 16, wherein when it is determined that the air bubbles are mixed in the expansion medium, the method further comprises:
acquiring, by the control unit, a second image data obtained by imaging the luminal organ while causing the pump to decompress the balloon;
comparing, by the control unit, the acquired second image data with first image data acquired while causing the pump to pressurize the balloon;
confirming, by the control unit, a change in size of the air bubbles mixed in the expansion medium; and
causing, by the control unit, the pump to perform the decompression operation before the preset decompression timing.

18. The indeflation method according to claim 17, wherein when it is determined that the air bubbles are mixed in the expansion medium, after acquiring the second image data, the method further comprises:
acquiring, by the control unit, as the first image data, image data obtained by imaging the luminal organ while causing the pump to pressurize the balloon.

19. The indeflation method according to claim 16, further comprising:
referring, by the control unit, to the first image data as the image data, when it is determined that the air bubbles are mixed in the expansion medium.

20. The indeflation method according to claim 16, further comprising:
determining, by the control unit, a possibility that air bubbles are mixed in the expansion medium by observing a change in pressure of the balloon while causing the pump to perform the pressurization operation;
decreasing, by the control unit, a speed of injecting the expansion medium into the balloon in the pressurization operation in a case where the determined possibility exceeds a reference; and
stopping, by the control unit, the pressurization operation when receiving a user operation requesting stop of the pressurization operation while causing the pump to perform the pressurization operation, and causing the pump to perform the decompression operation before the preset decompression timing even if it is not determined that the air bubbles are mixed in the expansion medium.
